(19) United States
(12) Patent Application Publication
(10) Pub. No.: US 2004/0152127 A1
(43) Pub. Date: Aug. 5, 2004

(54) PHOTOSENSITIVE MATERIALS AND METHOD FOR ACCELERATED DEVELOPMENT OF PHOTOSENSITIVE MATERIALS WITH TAILORED PROPERTIES FOR TAGGING OF OPTICAL MEDIA ARTICLES

(75) Inventors: Radislav Alexandrovich POTYRAILO, Niskayuna, NY (US); Marc Brian WISNUDEL, Clifton Park, NY (US)

Correspondence Address: FOLEY AND LARDNER, SUITE 500, 3000 K STREET NW, WASHINGTON, DC, 20007, US

(73) Assignee: GENERAL ELECTRIC COMPANY, Schenectady, NY (US)

(21) Appl. No.: 10/248,646

(22) Filed: Feb. 4, 2003

(51) Int. Cl. C12Q 1/68; G01N 33/53; G01N 33/543

(52) U.S. Cl. 435/7.1; 436/518

(57) ABSTRACT

A method of accelerated discovery of compositions that meet predetermined different requirements comprises: selecting a predetermined number of different dyes, electron donors and matrix forming materials; preparing a plurality of mixtures containing selected dyes, electron donors and matrix forming materials wherein each of the mixtures contains a different combination of dyes, electron donors and matrix forming materials; preparing an array of compositions by dissolving the mixtures in a solvent and applying the mixtures dissolved in the solvent onto one or more substrates; testing each of the compositions in the array for response to irradiation; compiling results of the testing into a data set; and classifying the data set with respect to a plurality of predetermined characteristics to form a classified data set.

### BACKGROUND OF THE INVENTION

The present invention relates to a method for accelerated development of photosensitive materials with tailored properties for tagging/identifying optical media articles.

Tagging of plastic articles is desirable in a number of different applications, including antipiracy protection of optical media. The use of tags in plastic materials is known in the art. For example, UV and near-IR fluorescent dyes have been added to polymers for identification purposes (see U.S. Pat. No. 4,238,524 issued on December 9, 1980 in the name of LaLiberte et al.; U.S. Pat. No. 5,005,873 issued on Apr. 9, 1991 in the name of West; U.S. Pat. No. 5,201,921 issued on Apr. 13, 1993 in the name of Luttermann et al.; U.S. Pat. No. 5,703,229 issued on December 30, 1997 in the name of Krutak et al.; U.S. Pat. No. 5,553,714 issued on September 10, 1996 in the name of Cushman et al.

The articles marked with the fluorophores include digital compact discs wherein the marking is use to determine their authenticity. An example of this technique is disclosed in U.S. Pat. No. 6,099,930 issued on Aug. 8, 2000 in the name of Cyr et al. According to this patent, a near infrared fluorophore can be incorporated into the CD by coating, admixing, blending or copolymerization. In addition to this, it is possible that more than one fluorophore is added to the polymer to enable the measure of a fluorescence ratio in the manner disclosed in British Patent Application publication GB-A-2264558 published on Sep. 1, 1993 to Theocharous.

However, this method suffers from the drawback that a shift in the ratio can occur in the event that any of the dyes ages or leaches under normal use conditions, and thus result in an erroneous identification. Causes of dye deterioration and/or concentration reduction include exposure to UV light, high ambient temperature, etc. In addition, additives in polymers can alter the ratio of fluorescence intensities. Fluorescence lifetime of an embedded dye has also been used for identification purposes—see U.S. Pat. No. 5,329,127 issued on Jul. 12, 1994 in the name of Becker et al. by way of example.

Therefore, a need still exists to be able to tag optical media and the like in a manner which will serve the intended purpose. However, there are a myriad of possible combinations of different materials which can be implemented in a variety of different ways. Accordingly, it is necessary to be able to rapidly analyze large numbers of combinations/possibilities and determine those which are practical and those which are not. A random approach to this analysis is however, unacceptable in that the amount of time consumed is untenably long.

### BRIEF SUMMARY OF THE INVENTION

A first aspect of the invention resides in a method of accelerated discovery of materials with predetermined properties comprising: selecting a plurality of first materials from a first class of materials; selecting at least one second material from a second class of materials; selecting a plurality of third materials from a third class of materials; preparing a plurality of mixtures which each contain one of the plurality of first materials, the at least one second material, and one of the plurality of third materials; forming an array of compositions by dissolving each of the mixtures in a solvent and applying each dissolved mixture onto a substrate; exposing the array of films to a plurality of predetermined environmental effects and recording variations in a plurality of predetermined characteristics in each of the films; exposing the array of films to at least one stimuli and recording variations in the plurality of predetermined characteristics in each of the films; compiling the recorded variations as a data set; and sorting the data set into predetermined categories wherein each category encompasses one of the predetermined characteristics.

A second aspect of the invention resides in a method of accelerated discovery of compositions that meet predetermined different requirements comprising: selecting a predetermined number of different dyes, electron donors and matrix forming materials; preparing a plurality of mixtures containing selected dyes, electron donors and matrix forming materials wherein each of the mixtures contains a different combination of dyes, electron donors and matrix forming materials; preparing an array of compositions by dissolving the mixtures in a solvent and applying the mixtures dissolved in the solvent onto one or more substrates; testing each of the compositions in the array for response to irradiation; compiling results of the testing into a data set; and classifying the data set with respect to a plurality of predetermined characteristics to form a classified data set.

A third aspect of the invention resides in a combinatorial chemistry method, comprising: providing an array of a plurality of different compositions; exposing the array to an external stimuli (stimulus) and determining two or more characteristics of a single property of the compositions.

### BRIEF DESCRIPTION OF THE DRAWINGS

FIG. 1 is a block diagram depicting a method for rapid high throughput, discovery and optimization of photosensitive material compositions in accordance with an embodiment of the invention.

FIG. 2 schematically depicts a high throughput screening cycle in accordance with an embodiment of the present invention.

FIG. 3 depicts arrays of fabricated compositions which are prepared in accordance with the method depicted in FIG. 1.

FIG. 4 schematically depicts screening results for photo-bleaching efficiency along with typical examples of spectral features under no bleaching, slight bleaching and strong bleaching, respectively.

FIG. 5 depicts screening results for recovery efficiency along with typical examples of recovery kinetics.

FIG. 6. shows screening results for bleaching rate along with typical examples of bleaching kinetics.

### DETAILED DESCRIPTION OF THE INVENTION

For tagging of optical media, it is advantageous to apply photosensitive compounds that change their optical properties upon interactions with a readout laser, for example at about 650 nm for DVD and at around 780 nm for CD readout. This tagging of optical media has multiple functions which include authentication, anti-piracy protection, and other functions. For these and other applications, the tagging materials must have a range of well defined optical properties. These properties can include nonreversible response with rapid photobleaching kinetics, nonreversible response with slow photobleaching kinetics, reversible response with rapid or slow on/off kinetics, and any combination of these parameters.

However, in order to sort through the very large number of possible material combinations via which these various parameters can be used/effectively implemented, it is necessary to implement accelerated discovery and optimization of material compositions that meet different requirements for different applications of photosensitive compounds in tagging of optical media articles.

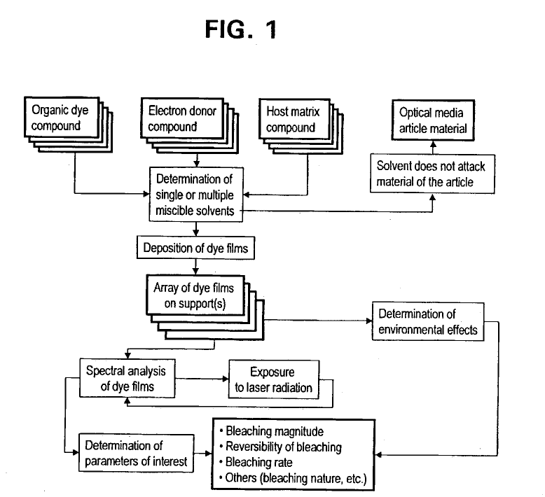

The first embodiment of the invention therefore includes a method for accelerated discovery and optimization of material compositions that meet different requirements for different applications of photosensitive compounds in tagging of optical media articles. A block diagram of main steps for discovery and optimization of these materials is depicted in FIG. 1.

A will be appreciated, a dye which is preferably an organic dye, is incorporated into a polymer host matrix by dissolving the dye and the matrix polymer in a single solvent or in a mixture of different but miscible solvents (viz., a solvent system). Optionally, other components are used in the composition. These components include but are not limited to electron donor materials such as triethanolamine, n-methyldiethanolamine, 2-{[2-(dimethyl(amino)ethyl]methyl-amino}-ethanol, tetramethylguanidine, tetra methylenethylene diamine, and many others. The solvent is selected on the basis that it does not attack or otherwise produce an injurious (detrimental) effect on the material of the optical media article during the time period necessary for the deposition and drying.

The dye/donor/polymer combinations used in the disclosed examples are deposited onto a single or multiple supports. The spectral analysis of the optical properties of the whole array of films is performed to determine the initial conditions of the films.

It should be noted that the films in this instance need not be homogenous in composition and can be produced by either mixing all of the components together and applying a coating which is allowed to dry to form a layer or film. Alternatively, the films can be formed by coating the materials (in a solvated state) individually one on top of each other so as to form a film or layer which is built up by the application of the different coats.

The films are exposed to laser radiation with the laser wavelength corresponding to the intended operation of the optical media article. The spectral properties of the films are analyzed after the exposure to determine a variety of relevant parameters of interest. The data is collected and compiled into a data set. The parameters of interest include but are not limited to bleaching (viz., decolorization) magnitude, reversibility of bleaching (viz., recolorization), bleaching rate (decolorization rate), and any others (for example, bleaching/decolorization nature/characteristics, etc.).

It will be appreciated that, in order to distinguish between a real and fake item it is often necessary to determine not only that decolorization (for example) occurs but also the rate of change or the degree to which the change occurs. By having at least two characteristics of a single parameter it is possible to improve the ability with this real and fake items or articles can be distinguished from each other. Different layers can be used. For example, as will be disclosed hereinlater, it is possible to use a binary system wherein an upper layer must be bleached or decolorized (for example) before an underlying layer can be irradiated to produce a given rate of colour change (for example). The design of such system can be very rapid given the data which is rendered possible with the first embodiment of the invention.

A second embodiment of the invention comprises an authenticate-able media and method for manufacturing media involving: 1) initially coating media or molding media with dye-dispersed polycarbonate such that the dye covers or is in the substrate, followed by 2) a photo-mask operation that effectively removes via photobleaching un-wanted dye in or on the media resulting in spatially-resolved patterns or spots.

### EXAMPLES

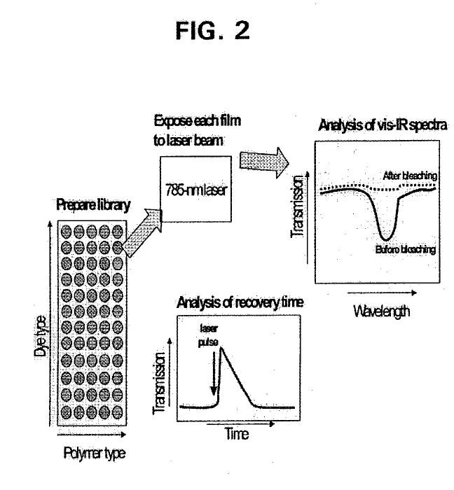

Compositions that were photoresponsive to the laser radiation in the range of 780-785 nm from a CD ROM drive were determined. For these determinations, 12 dyes, five polymer matrices, and one electron donor were selected. Thus, 12×1×5=60 film compositions were made to determine dye/donor/polymer interactions. The high throughput screen was performed when the films were arranged as 48-element film arrays and were exposed to a 785-nm laser. The spectral analysis was performed using an automated spectroscopic setup. The screening cycle is depicted in FIG. 2.

The polymers used in this example are listed in Table 1. The dyes used are listed in Table 2. As an electron donor, triethanolamine was used. Other electron donors can be n-methyidiethanolamine, 2-{[2-(dimethyl(amino)ethyl]methyl-amino}-ethanol, tetramethylguanidine, tetra methylenethylene diamine, and many others.

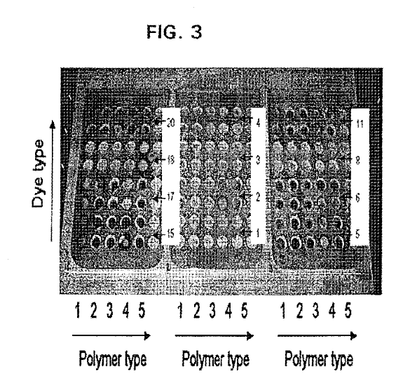

Stock solutions of dissolved polymers were made by dissolving polymers either in ethanol or water at a concentration of 30% wt. Stock solution of electron donor was made by dissolving triethanolamine in water at a concentration of 30% wt. Next, a stock solution of polymer/electron donor was made with 2/3 of the polymer stock solution and 1/3 of electron donor stock solution. The dyes listed in Table 2 were dissolved in ethanol at a concentration approaching their saturation level. Finally, 450 microliters of the polymer/electron donor solutions were mixed with 100-200 microliters of the dye solutions. These dye solutions were deposited (about 30 microliter volumes) into the wells formed in polycarbonate substrates. The solutions were allowed to dry overnight at room temperature. For laser bleaching, an SDL laser emitting at 785 nm was used. FIG. 3 illustrates all fabricated libraries of 60 compositions which were each made in duplicate.

TABLE 1
Polymers

| Code | Polymer type |
|---|---|
| 1 | PVOH poly(vinyl alcohol) |
| 2 | PVPD poly(vinyl pyrrolidone) |
| 3 | HPC hydroxypropyl cellulose |
| 4 | Nafion |
| 5 | PSS polystyrene sulfonate Na salt |

TABLE 2
Dyes

| Code | Dye type |
|---|---|
| 1 | LC 1090 |
| 2 | LC 8000 |
| 3 | LC 1080 |
| 4 | DCCP diethylthiatricarbocyanine perchlorate |
| 5 | IR 125 |
| 6 | Cryptocyanine |
| 8 | Octabutoxycyanine |
| 11 | cresyl violet acetate |
| 15 | janus green B |
| 17 | SDA 6995 |
| 18 | Malachite green |
| 20 | Methylene blue |

Screening Results for Photo-Bleaching Efficiency

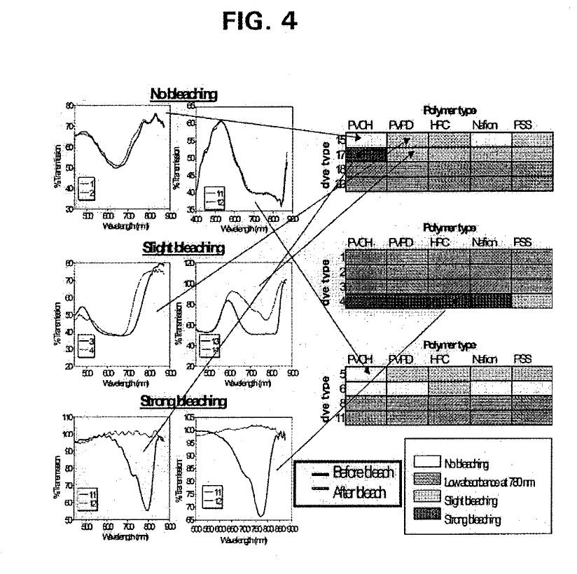

Screening results for photo-bleaching efficiency are presented in FIG. 4. The strong bleaching with the 785 nm radiation was observed with several compositions indicated in the illustrated manner. Typical examples of spectral features under no bleaching, slight bleaching and strong bleaching are also depicted in FIG. 4.

Screening Results for Recovery Efficiency

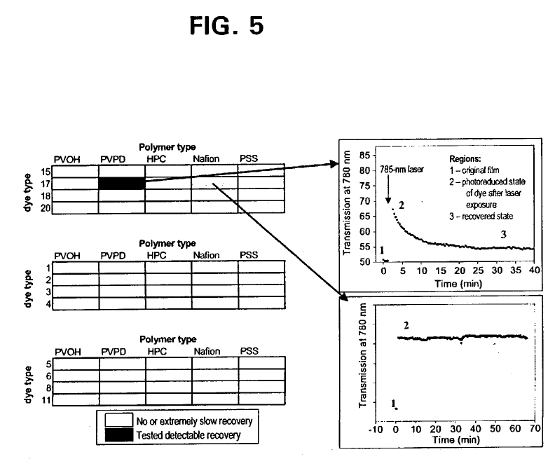

Screening results for recovery efficiency are presented in FIG. 5. The recovery of transmission after the 785 nm radiation exposure was observed with a PVPD/SDA 6995 composition. Typical examples of spectral features wherein detectable recovery and no recovery are also depicted in FIG. 5.

Screening Results for Bleaching Rate

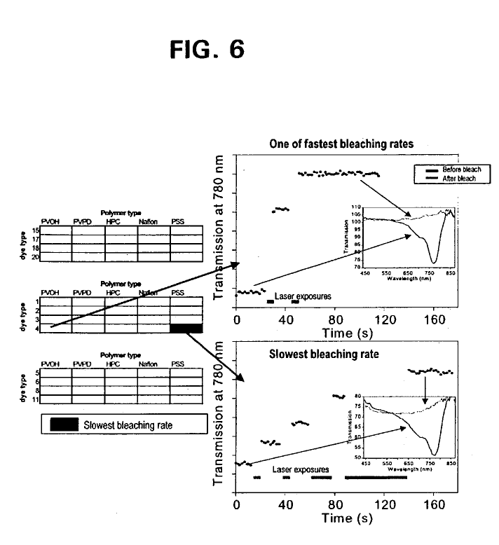

Screening results for bleaching rate are presented in FIG. 6. The bleaching rate under the 785 nm radiation exposure was the slowest for the observed with a PSS/DCCP composition. Typical examples of bleaching rate are also depicted in FIG. 6. Methods for dye incorporation into optical media are depicted in FIGS. 7-10.

For example, as shown in FIG. 7 shows a photo-mask process for creating spatially-resolved patterns or spots on or in media from substrates that are initially coated or molded from dye-dispersed resins. As will be appreciated, there are a number of locations and/or methods via which a dye which has been determined using the above disclosed technique can be disposed. The dyes can be arranged to change from transparent to opaque and are incorporated into a photosensitive compound. These can be arranged to initially not absorb laser energy.

FIGS. 8A and 8B respectively depict a scan of coated DVD after a photo-masking process; and a spatially-resolved reflectivity change at 650 nm measured across the a ring resulting from photobleaching through a mask.

FIG. 9 shows examples of how the dye can be arranged to convert from an opaque state to a transparent state. This can be used using a photomask coating approach. The dye can be disposed in the photosensitive compound. Irradiation is used with the photomask to eliminate inverse of spot. That is to say, create micro dye from macro processes of molding and/or spin-coating.

A surface modified coating approach may be used. With this technique the entire surface is coated using spin-coating. However, the coating only sticks to areas that have been pretreated with UV. This technique requires a differentiated photosensitive compound with a modified polarity or surface energy, e.g. additives, endcaps or copolymer.

A binary approach is such that the compound in the photosensitive compound is used in conjunction with Coated spot. For example near-IR absorbers in combination with a thermochromic compound.

Other optical properties can include photosensitive compounds modified to improve media performance (refractive index, laser sensitivity, color to block undesirable light (photobleach resistance), etc), or for aesthetic purposes such as to provide color in a photosensitive compound to hide authentication spots from hackers and also to differentiate products from one another.

What is claimed is:

1. A method of accelerated discovery of materials with predetermined properties comprising:
selecting a plurality of first materials from a first class of materials;
selecting at least one second material from a second class of materials;
selecting a plurality of third materials from a third class of materials;
preparing a plurality of mixtures which each contain one of the plurality of first materials, the at least one second material, and one of the plurality of third material;
forming an array of compositions by dissolving each of the mixtures in a solvent and applying each dissolved mixture onto a substrate;
exposing the array of films to a plurality of predetermined environmental effects and recording variations in a plurality of predetermined characteristics in each of the films;
exposing the array of films to at least one stimuli and recording variations in the plurality of predetermined characteristics in each of the films;
compiling the recorded variations as a data set; and
sorting the data set into predetermined categories wherein each category encompasses one of the predetermined characteristics.

2. A method as set forth in claim 1, wherein the first class of materials comprises dyes.

3. A method as set forth in claim 1, wherein the second class of materials comprises electron donor materials.

4. A method as set forth in claim 1, wherein the third class of materials comprise host matrix forming materials.

5. A method of accelerated discovery of compositions that meet predetermined different requirements comprising:
selecting a predetermined number of different dyes, electron donors and matrix forming materials;
preparing a plurality of mixtures containing selected dyes, electron donors and matrix forming materials wherein each of the mixtures contains a different combination of dyes, electron donors and matrix forming materials;
preparing an array of compositions by dissolving the mixtures in a solvent and applying the mixtures dissolved in the solvent onto one or more substrates;
testing each of the compositions in the array for response to irradiation;
compiling results of the testing into a data set; and
classifying the data set with respect to a plurality of predetermined characteristics to form a classified data set.

6. A method as set forth in claim 5, wherein the irradiation comprise laser irradiation.

7. A method as set forth in claim 5, wherein the predetermined characteristics comprise at least one of, decolorization, recolorization, rate of decolorization, rate of recolorization, degree of decolorization, degree of recolorization, and permanency of decolorization.

8. A method as set forth in claim 5, further comprising selecting a combination of dye, electron donor and host matrix material based on the data.

9. A method as set forth in claim 8, comprising applying the selected combination of dye, electron donor and host matrix to an article in a manner which renders the article identifiable via irradiation of the article with a laser having a predetermined wavelength.

10. A method as set forth in claim 5, further comprising:
forming a film containing a dye, electron donor and host matrix material selected based on the classified data set, on an article;
masking the film;
exposing unmasked portions of the film to an exposure stimulus selected from among the predetermined stimuli to cause a change in the unmasked portions of the film;
removing the unmasked portion of the film which has been changed by the exposure to the exposure stimulus.

11. A method as set forth in claim 10, further comprising using the film to produce a unique measurable characteristic via which the article on which the film is formed can be identified via exposure to a stimulus selected from among the plurality of predetermined stimuli.

12. A combinatorial chemistry method, comprising:
providing an array of a plurality of different compositions;
exposing the array to an external stimuli (stimulus) and determining two or more characteristics of a single property of the compositions.

13. A method as set forth in claim 12, wherein the plurality of compositions comprise a host matrix, dye and electron donor compositions.

14. A method as set forth in claim 12, wherein the array of different composition comprise an array of films of different compositions.

15. A method as set forth in claim 12, wherein the array of different composition comprise an array of films of different mixtures.

16. A method as set forth in claim 13, wherein the single property comprises a reaction to laser irradiation and the two or more characteristics are selected from decolorization, recolorization, rate of decolorization, rate of recolorization, degree of decolorization, degree of recolorization, and permanency of decolorization.

17. A method as set forth in claim 16, wherein decolorization comprises a magnitude of change from one state to another due to photoreduction from laser irradiation.

18. A method as set forth in claim 16, further comprising further comprising:
compiling the determined characteristics into a data set; and
selecting a combination of dye, electron donor and host matrix material based on the data.

19. A method as set forth in claim 18, comprising applying the selected combination of dye, electron donor and host matrix to an article in a manner which renders the article identifiable via irradiation of the article with a laser having a predetermined wavelength.

20. A method as set forth in claim 17, further comprising:
dissolving a plurality of combinations of different host matrix material, electron donor and dye materials in one or more solvents to form a plurality of mixtures of different compositions;
applying the plurality of mixtures onto one or more substrates; and
determining if any of the mixtures detrimentally effects the one or more substrates.
